(19) United States
(12) Patent Application Publication
(10) Pub. No.: US 2021/0100616 A1
(43) Pub. Date: Apr. 8, 2021

(54) SYSTEMS AND METHODS FOR PLANNING PERIPHERAL ENDOVASCULAR PROCEDURES WITH MAGNETIC RESONANCE IMAGING

(71) Applicant: Sunnybrook Research Institute, Toronto (CA)

(72) Inventors: Trisha Roy, Toronto (CA); Andrew Dueck, Toronto (CA); Graham A. Wright, Toronto (CA); Garry Liu, Toronto (CA)

(21) Appl. No.: 16/500,109

(22) PCT Filed: Apr. 5, 2018
(86) PCT No.: PCT/CA2018/050420

§ 371 Date: Oct. 2, 2019

Related U.S. Application Data
(60) Provisional application No. 62/481,899, filed on Apr. 5, 2017.

(51) Int. Cl. A61B 34/10; G01R 33/56; G01R 33/561; G01R 33/563; G01R 33/483; G01R 33/54; A61B 5/00; A61B 5/107

(52) U.S. Cl. CPC A61B 34/10; G01R 33/5608; G01R 33/5614; G01R 33/5635; A61B 2034/105; G01R 33/546; A61B 5/004; A61B 5/1075; A61B 5/1079; G01R 33/483

(57) ABSTRACT

Systems and methods for planning peripheral endovascular, and other, procedures based on magnetic resonance imaging (“MRI”] are provided. Mechanical properties of lesions, morphology, and vessel patency are characterized based on non-contrast angiography and ultrashort echo time (“UTE”] images. The methods described in the present disclosure also provide improved visualization of the vascular tree and microchannels.

### CROSS-REFERENCE TO RELATED APPLICATIONS

This application claims the benefit of U.S. Provisional Patent Application Ser. No. 62/481,899, filed on Apr. 5, 2017, and entitled “SYSTEMS AND METHODS FOR PLANNING PERIPHERAL END OVASCULAR PROCEDURES WITH MAGNETIC RESONANCE IMAGING,” which is herein incorporated by reference in its entirety.

### BACKGROUND

There are two treatment options for revascularizing patients with critical limb ischemia: bypass surgery and percutaneous vascular intervention (“PVI”). PVI is less invasive but has high immediate technical failure rates (20%) and high re-intervention rates (20%). The most common mode of immediate failure is the inability to enter/cross the lesion.

With current imaging (x-ray angiography, CTA, duplex ultrasound) it is difficult to predict which lesions will be soft enough to cross with a wire to make PVI possible. Physicians have responded with a “percutaneous-first” strategy where they attempt PVI in all patients and perform surgery if PVI fails. This requires more procedures per index limb at significant cost to healthcare systems and delays definitive revascularization. Additionally, there is evidence that surgical bypass after failed PVI results in worse outcomes, including higher amputation rates within 1 year.

These issues highlight the need for improved diagnostic accuracy to inform patient selection.

Current clinical imaging modalities are primarily “lumenography” techniques that demonstrate only length, degree of stenosis/occlusion, stump morphology, and presence of calcium. These parameters have important implications as longer lesions, chronic total occlusions (CTOs), blunt stump morphology, and calcified lesions have higher failure rates. This gross characterization facilitates some treatment decisions, such as choosing a hybrid approach with femoral endarterectomy for a heavily calcified common femoral artery or choosing not to stent long lesions that cross a joint to prevent kinking. However, the length and degree of stenosis/occlusion of a lesion is not equivalent to its burden, mechanical properties or morphology, all of which influence PVI success.

There have been recent advances in invasive vascular imaging plaque characterization techniques. These include virtual histology intravascular ultrasound (IVUS) with automated plaque segmentation, optical coherence tomography, and angioscopy, that are able to characterize concentric versus eccentric plaque, calcium morphology, lipid-rich versus fibrous plaques, fibrous cap thickness, macrophage infiltration, and even thrombus types and age. These plaque characteristics influence the success of various treatment modalities. Intravascular imaging devices, however, require invasive arterial access which makes the “percutaneous-first” strategy and associated complications impossible to avoid. The added procedure time and cost of intravascular imaging devices also limit their widespread clinical use, which provides motivation to improve non-invasive lesion characterization imaging.

Noninvasive imaging modalities, including computed tomography (CT) and magnetic resonance imaging (MRI), are an area of intense research. The primary focus for plaque characterization research thus far has been the identification of high-risk, vulnerable plaques in the carotid and coronary arteries. These techniques are optimized to identify lipid rich necrotic cores, which are a key feature in carotid and coronary arteries, and can predict stroke or myocardial infarction. The pathogenesis of peripheral arterial disease is multifactorial, but there is evidence to suggest that the majority of peripheral arterial disease is arteriosclerotic, not atherosclerotic. The primary disease pattern involves medial wall calcification from a mechanism that is independent of atherosclerotic plaque development. Existing plaque analysis techniques with CT or MRI are tailored to characterize atherosclerotic plaque, but are not tailored to characterize peripheral arterial lesions, specifically. 100081 Though the mechanical properties of atherosclerotic plaques have been described, the prognostic value of mechanical properties for planning endovascular treatment has not been comprehensively investigated. Ultrasound elastography is one technique that has related the mechanical properties of hard versus soft lesions in peripheral arteries and endovascular procedural outcomes. However, ultrasound elastography is limited due to issues with severely calcified vessel acoustic shadowing, repeatability and user dependence, penetration depth, and inability to perform 3D lesion analysis.

It is challenging to accurately evaluate heavily calcified small-caliber vessels using non-invasive techniques, including CTA and duplex ultrasound.

### SUMMARY OF THE DISCLOSURE

The present disclosure addresses the aforementioned drawbacks by providing a method for characterizing a lesion in a subject using magnetic resonance imaging (“MRI”). Magnetic resonance images acquired from a volume-of-interest in a subject, first echo time images acquired from the volume-of-interest in the subject using a first echo time that is in a range of ultrashort echo times, and second images acquired from the volume-of-interest in the subject using a second echo time that is longer than an ultrashort echo time are provided to a computer system. Combined images are produced by computing a mathematical combination of the first images and the second images. A lesion is identified in the magnetic resonance images, and the mechanical properties of the identified lesion are characterized based at least in part on a comparison of magnetic resonance signal behaviors between the magnetic resonance images and the combined images.

It is another aspect of the present disclosure to provide a method for generating an endovascular procedure plan using MRI. Magnetic resonance angiography images acquired from a volume-of-interest in a subject, first images acquired from the volume-of-interest in the subject using a first echo time that is in a range of ultrashort echo times, and second images acquired from the volume-of-interest in the subject using a second echo time that is longer than an ultrashort echo time are provided to a computer system. A three-dimensional angiogram is generated from the magnetic resonance angiography images. The three-dimensional angiogram depicts the vasculature of the subject. Combined images are produced by computing a mathematical combination of the first images and the second images. A lesion is identified in the magnetic resonance angiography images. Fusion image data are generated based on a combination of the magnetic resonance angiography images and the combined images, wherein the fusion image data provides a characterization of the identified lesion. The fusion image data and the three-dimensional angiogram are then processed to generate a report that indicates an endovascular procedure plan for the subject.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flowchart setting forth the steps of an example method for characterizing lesion hardness, assessing vessel occlusion or patency, generating a treatment plan for an interventional procedure, and so on, using the methods described in the present disclosure.

FIG. 2A shows in situ combined images generated as difference images by computing a difference between an ultrashort echo time image and a longer echo time image.

FIG. 2B shows ex vivo images of three different lesion morphologies, include combined images, microCT images, and histology images.

FIG. 3 shows examples of plaque characteristics on combined images, which can be used to characterize hard and soft lesion components.

FIG. 4 shows an example fusion image data set in which a hard, but non-calcified, lesion can be visualized, where that lesion cannot be visualized with x-ray angiography.

FIGS. 5A-5E illustrate example images in a fusion image data set that can be used for assessing patent and occluded vessels.

FIGS. 6A-6F show various aspects of a user interface that can be implemented on a hardware processor and a memory to provide user input that can operate the hardware processor to implement methods described in the present disclosure.

FIG. 7 is a block diagram of an example computer system that can implement the methods described in the present disclosure.

FIG. 8 is a block diagram of an example image processing unit implemented with at least one hardware processor and at least one memory, which can implement the methods described in the present disclosure.

FIG. 9 is a block diagram of an example magnetic resonance imaging (“MRI”) system that can implement the methods described in the present disclosure.

### DETAILED DESCRIPTION

Described here are methods for characterizing the mechanical properties lesions or other regions of tissue, as well as assessing patency, using magnetic resonance imaging (“MRI”). Such methods can be implemented for planning peripheral endovascular, or other vascular, procedures. The methods described in the present disclosure include acquiring magnetic resonance images using different contrast weightings and analyzing those images together in a single analytical framework to characterize properties of the subject's vasculature. The properties that can be characterized include patency (e.g., the degree of stenosis, occlusion, or both), mechanical properties (e.g., stiffness, which can be used to differentiate hard plaque components from soft plaque components), tissue content (e.g., calcification content, collagen content), and morphology (e.g., eccentricity, stump morphology). The methods described in the present disclosure also provide improved visualization of the vascular tree and microchannels.

In general, the methods described in the present disclosure can include generating a flow-independent angiogram based on magnetic resonance images; detecting lesions or other relevant regions of tissue based on magnetic resonance images; characterizing the mechanical properties of the detected lesions or other relevant regions of tissue; and generating a fused image data set that can be used to display a map of mechanical properties of target lesions. The fused image data set can also be used to identify microchannels and soft lesion components that may facilitate wire passage. The composition and morphology of target lesions can also guide wire and device selection for peripheral endovascular procedures.

In some aspects, the present disclosure provides methods for flow-independent MRI that can be used to generate flow-independent angiograms. Flow-independent imaging enables more accurate imaging of small caliber occlusive peripheral vessels with variable velocity of blood flow. This technique allows for the identification of microchannels and intermittent patencies that are not seen with x-ray angiography, which is the current gold standard for producing angiograms.

The flow-independent imaging displays both the lumen and vessel wall to make more accurate measurements than conventional lumenography imaging. This facilitates the selection of wires with appropriate calibers and enables more accurate sizing for balloons and stents.

In some other aspects, the present disclosure provides methods that can accurately differentiate hard lesion components, whether calcified or non-calcified, from soft lesion components, and can characterize lesion morphology. These features inform procedure planning because hard lesions may be more suitable for bypass surgery or require specialized stiff wires. Morphology also affects planning. As an example, concentric lesions are less amenable to drug-eluting therapy compared with eccentric lesions.

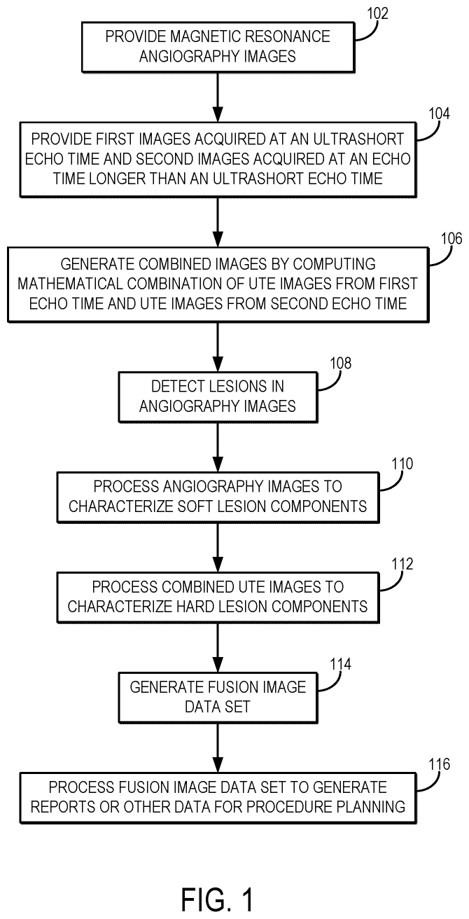

Referring now to FIG. 1, a flowchart is illustrated as setting forth the steps of an example method for creating or updating an interventional procedure plan, such as a peripheral endovascular procedure, based on magnetic resonance imaging.

The method includes providing magnetic resonance angiography images to a computer system, as indicated at step 102. In some embodiments, these images can be flow-independent angiography images acquired without the use of an exogenous contrast agent (e.g., gadolinium) and, as such, can be referred to as non-contrast enhanced angiography images. From these images, angiograms can be generated as is known in the art. Providing the angiography images can include retrieving previously acquired images from a memory or other data storage, or can include acquiring such images from a subject using an MRI system.

In general, the angiography images are images acquired without an exogenous contrast agent (e.g., a gadolinium-based contrast agent), but depict sufficient image contrast in the subject's vasculature to provide angiographic information. For instance, in such images flowing blood may appear hyperintense (i.e., bright) such that signals from blood can be readily identified both visually and for processing.

As one example, the angiography images can be acquired using a steady-state free precession (“SSFP”) pulse sequence. In one non-limiting example, the SSFP pulse sequence can be a balanced binomial-pulse SSFP (“BP-SSFP”) pulse sequence, such as the one described by G. Liu, at al. in “Balanced Binomial-Pulse Steady-State Free Precession (BP-SSFP) for Fast, Inherently Fat Suppressed, Non-Contrast Enhanced Angiography,” Proc Intl Soc Mag Reson Med, 2010; (18):3020. Such pulse sequences are beneficial for creating flow-independent angiograms because they do not require the use of an exogenous contrast agent, which in turn allows these pulse sequences to be used for imaging slow-flowing occlusive run-off arteries. As a result, these pulse sequences can be used to identify stenoses, occlusions, or both, and to assess run-off vessels, such as tibial run-off vessels in the peripheral vasculature.

As one non-limiting example, angiography images can be obtained using a 3D BP-SSFP pulse sequence with the following parameters: field-of-view of 24×24×24 cm³, image resolution of 1×1×1 mm³, repetition time (“TR”) of 5.54 ms, echo time (“TE”) of 3.58 ms, flip angle of 45 degrees, number of averages (“NEX”) of 1, and a total acquisition time of 2 minutes.

Referring still to FIG. 1, the method also includes providing images acquired using an ultrashort echo time (“UTE”) pulse sequence, as indicated at step 104. In general, a UTE pulse sequence will include at least one echo time that is in a range of ultrashort echo times, such that tissues with short T₂ or T*₂ values can be imaged, and a second echo time that is longer than an ultrashort echo time. In general, ultrashort echo times can include echo times that are shorter than 1 millisecond. In other examples, ultrashort echo times can be selected as less than 500 microseconds. In other implementations, zero echo time (“ZTE”) or sweep imaging with Fourier transform (“SWIFT”) pulse sequences could also be used to acquire images that depict tissue with short T₂ or T*₂ values. In any event, providing these images can include retrieving previously acquired images from a memory or other data storage, or can include acquiring such images from a subject using an MRI system.

The images acquired with a UTE pulse sequence can generally be referred to as UTE images, and generally include images acquired at two different echo times. The first echo time occurs very shortly after the RF excitation, such as in a range of ultrashort echo times. As one non-limiting example, the first echo time can be on the order of tens of microseconds or less. Data acquired at this first echo time will still include magnetic resonance signals from tissues or other materials with short T₂ or T*₂ values (e.g., calcium, collagen). The second echo time occurs longer after the RF excitation, such as at an echo time that is longer than an ultrashort echo time. As one non-limiting example, this second echo time can be on the order of a few milliseconds after RF excitation. Data acquired at this second echo time will include fewer magnetic resonance signals from those tissues or other materials with short T₂ or T*₂ values since those signals rapidly decay. The second echo time can be selected to account for the fat-water chemical shift at the magnetic field strength of the MRI system. The UTE images thus include first images associated with data acquired at the first, ultrashort echo time, and second images associated with data acquired at the second echo time, which is longer than an ultrashort echo time.

As one non-limiting example, a UTE pulse sequence with the following parameters can be used: field-of-view of 18×18×18 cm³ , image resolution of 1×1×1 mm³, TR of 10 ms, a first TE (“TE1”) of 30 μs and a second TE (“TE2”) of 2.25 ms, flip angle of 9 degrees, number of averages (“NEX”) of 1, and a total acquisition time of 15.5 minutes. The second echo time was selected to account for the fat-water chemical shift at 3T, which improves the emphasis of short-T2 signal components in the subtraction of images acquired at the first echo time and images acquired at the second echo time.

Referring still to FIG. 1, combined images are computed by computing a mathematical combination of the images acquired at the first, ultrashort, echo time (e.g., the first images) and the images acquired at the second echo time (e.g., second images), which is longer than an ultrashort echo time, as indicated at step 106. The mathematical combination could be a linear combination or a non-linear combination. As one example, computing the mathematical combination can include computing a difference, which may be a weighted or non-weighted difference, between the first and second images. As another example, the mathematical combination can be a non-linear combination, in which T*₂ is estimated based on a combination of the first and second images. These combined images depict short-T2 signal components, such as calcium and collagen. Window and leveling of these combined images can be determined based on a reference tissue. As one example, the reference tissue can be the intermuscular fascia. In this example, the window level can be set as the mean signal intensity of the intermuscular fascia, and the window width can be set as twice the window level.

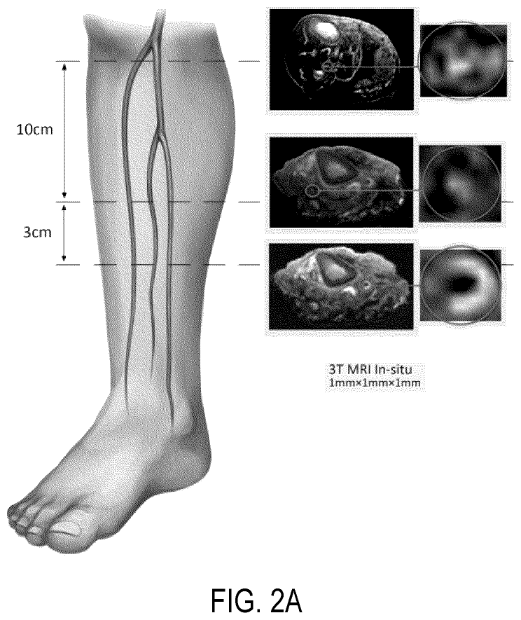

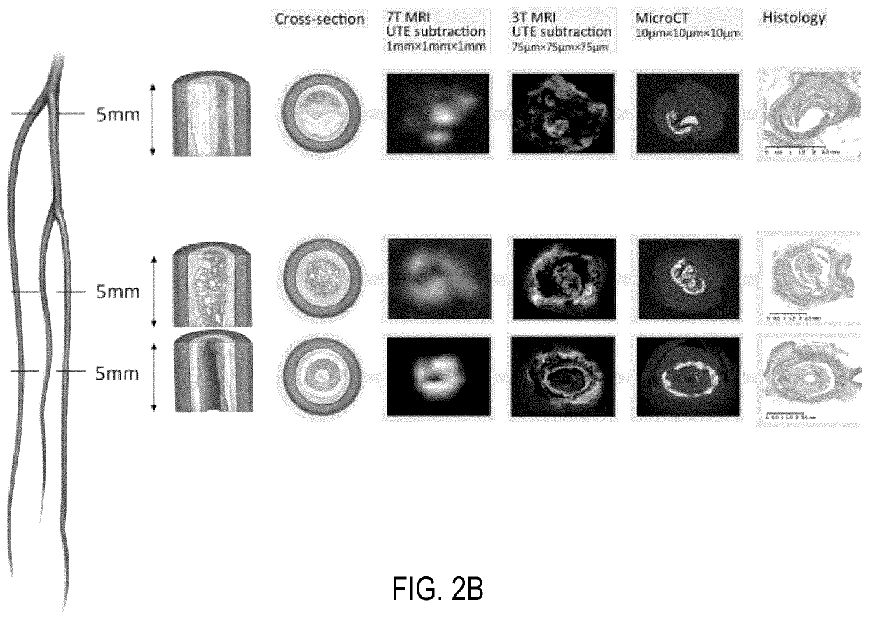

Examples of validation images of three different lesion morphologies are shown in FIGS. 2A (in situ) and 2B (ex vivo), which depict combined images generated as difference images. The lesion morphology shown in the top row of FIG. 2B is a calcium nodule within soft matrix. The lesion morphology in the middle row of FIG. 2B is speckled calcium intermixed with collagen. The lesion morphology in the bottom row of FIG. 2B is concentric medial calcium around soft matrix and a microchannel.

Referring again to FIG. 1, lesions are then detected in the angiography images, as indicated at step 108. For example, lesions can be automatically detected by detecting signal drop-out in bright-blood angiograms. The detected lesions are then analyzed to characterize soft lesion components and hard lesion components. The location of the lesions in the angiography images can be associated with the UTE images by co-registering the images.

In general, the angiography images can be processed to characterize soft lesion components for a detected lesion, as indicated at step 110. Previous studies have shown that lesions composed primarily of thrombus, soft proteoglycan matrix, microchannels, or combinations thereof, require low guidewire puncture forces. These soft lesions are likely more amenable to endovascular treatment.

The MRI signal behavior of thrombus can vary with age. It is contemplated that acute thrombus may appear hyperintense on T1-weighted and T2-weighted images, and may reach peak intensity at approximately one week before decreasing in signal intensity to a plateau at approximately six weeks. This change in signal intensity may depend on the ferric iron content in the thrombus, which can have a dephasing effect that shortens T2 signal decay times. SSFP pulse sequences have a mixed T1 and T2 weighting, in which it is contemplates that an aged thrombus may appear to have little signal. During UTE imaging, the thrombus signal intensity may not vary significantly between the two echo times and, thus, may subtract out in combined UTE images computed as difference images.

Referring still to FIG. 1, the UTE images (e.g., the first images from the ultrashort echo time, the second images from the longer echo time, the combined images, or combinations thereof) can be processed to characterize hard lesion components for a detected lesion, as indicated at step 112. Previous studies have shown that lesions composed primarily of collagen and calcium require high guidewire puncture forces that can exceed the tip load of clinically available guidewires. These hard lesions would likely be at higher risk of endovascular failure.

Calcium and collagen have very short T2 decay times. As a result, they produced signal only at the early echo time of a UTE pulse sequence (i.e., in the UTE images acquired at the first, ultrashort echo time). At the later echo time in the UTE pulse sequence, the calcium and collagen signal will have decayed significantly. Other tissue components including skeletal muscle, smooth muscle around the vessel wall, fat, or flowing blood have slower T2 decay times that do not vary significantly between the first and second echo times used in the UTE pulse sequence. Thus, when the difference between the first and second images is computed, tissues with slow T2 decay times are effectively subtracted out and tissues with short T2 decay times (e.g., calcium and collagen) are highlighted in the resulting combined images.

The general signal behaviors for various tissue types that may be encountered when imaging the peripheral vasculature are summarized in Table 1 below.

TABLE 1
MRI Signatures of Peripheral Artery Disease Lesion Components

| Plaque Component | T2-Weighted Images | UTE Images |
|---|---|---|
| Fat | Hyperintense | Hyperintense |
| Thrombus | No signal | Hyperintense |
| Soft Tissue | Isointense | Isointense |
| Smooth Muscle | Reference Intensity | Reference Intensity |
| Hardened Tissue | Hypointense | Isointense |
| Calcium | No signal | Hypointense |
| Lumen | No signal | No signal |

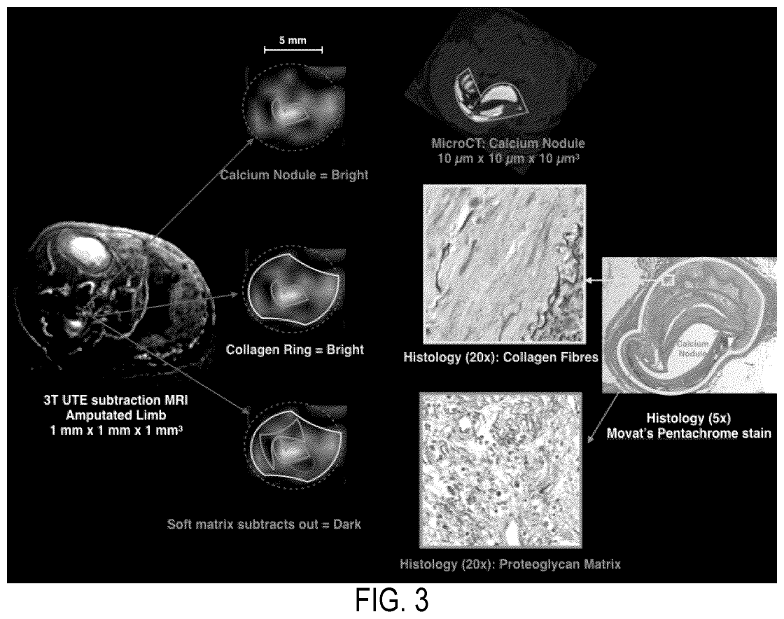

FIG. 3 shows an example in which combined images are used to assess or otherwise characterize lesion components. Even at 1 mm isotropic resolution, it is possible to differentiate hard plaque components (e.g., calcium nodule and collagen ring) from soft plaque components (e.g., proteoglycan matrix). The calcium nodule can be seen on both MRI and microCT. The collagen ring can only be visualized with MRI.

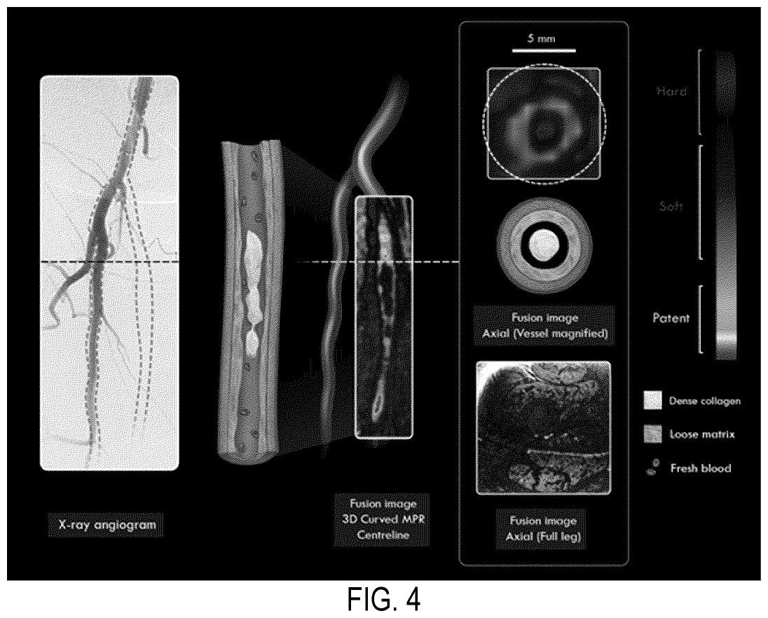

Referring again to FIG. 1, a fusion image data set can be generated based on the angiography images and the UTE images (e.g., the first images from the first, ultrashort echo time, the second images from the second, longer echo time, the combined images, or combinations thereof), as indicated at step 114. In some instances, the fusion image data set can be colorized for easier interpretation by a user. An example of a hard, but non-calcified, lesion that can be visualized with the fusion image data set, but not with x-ray angiography, is shown in FIG. 4.

In some embodiments, the fusion image data set can be generated as a pixel-wise, or voxel-wise, tissue classification map derived from signals in the input images. As one example, a statistical pattern recognition technique, such as fuzzy clustering, can be performed on the signals from the input images to generate a vector for each pixel, or voxel, for the fusion image data sets. As another example, deep learning algorithms can be applied to the input images, where such learning algorithms are trained to determined hard lesion components versus soft lesion components based on signal patterns in the input images. Persons having ordinary skill in the art will appreciate that the steps of characterizing the lesions (steps 110 and 112) can thus be performed based on fusion image data sets. In these instances, steps 110 and 112 may be performed after the fusion image data set has been generated.

The fusion image data set can then be processed, as indicated at step 116, to provide one or more reports on a patient-specific plan for an interventional procedure, including which procedure may be most effective for the patient based on the assessment of the disease state of the vasculature. 100491 In some aspects, processing the fusion image data set can include generating a map of mechanical properties of one or more detected lesions, where the mechanical properties of the lesions can be derived by their magnetic resonance signal behavior and relative signal intensities. The methods described in the present disclosure differ from previous techniques for characterizing mechanical properties of lesions, which are based on elastography or computational models that use finite element, fluid dynamics, or other quantitative modeling techniques.

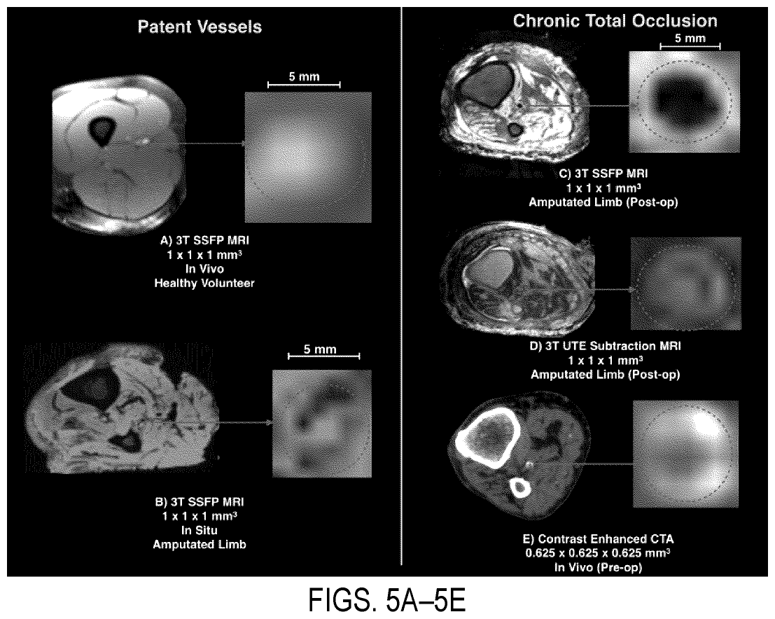

In some other aspects, processing the fusion image data set can include analyzing the images contained therein to assess vessel occlusion or patency, which may be used to determine or otherwise inform an optimal pathway for a given procedure to reach a treatment region, such as a region containing a lesion. Examples of patent versus occluded vessels, which can be detected with the methods described in the present disclosure, are shown in FIGS. 5A-5E. FIG. 5A shows a healthy volunteer showing bright signal in the superficial femoral artery. FIG. 5B shows a diseased, but patent, tibial artery within an amputated limb, which also demonstrates bright signal in the lumen of the artery. FIGS. 5C-5E show popliteal chronic total occlusion. FIG. 5C shows that the popliteal artery appears dark in the SSFP image (e.g., an angiography image) indicating that an occlusion is present. This technique does not suffer from calcium blooming and provides a sharper outline of the lesion. FIG. 5D shows that the occlusion is characterized as “hard” because it is bright on a combined image. FIG. 5E shows an example of a preoperative CT angiography image that shows a concentric calcium ring that correlates with the combined UTE image. CT angiography suffers from calcium blooming making it difficult to evaluate the degree of stenosis. CTA does not show the occlusive plug of collagen seen on combined images.

Processing the fusion image data set can also include plotting signal intensities from the various images in the fusion image data set against each other and using a clustering algorithm to separate tissue types of different compliances depending on their signal behavior.

In some aspects, processing the fusion image data set can include processing the fusion image data set to identify a recommended path to navigate through soft lesion components, microchannels, and patencies.

In some other aspects, processing the fusion image data set can include processing the fusion image data set to identify one or more angiosomes. In general, an angiosome is an anatomic unit of tissue (e.g., containing skin, subcutaneous tissue, fascia, muscle, and bone) fed by a source artery and drained by specific veins. Thus, processing the fusion image data set to identify an angiosome can also include identifying the feeder artery associated with the angiosome. The ability to identify an angiosome and its associated feeding artery can benefit interventional procedures, such as by identifying the vasculature that should be targeted for revascularization, which may include identifying one or more alternative paths for revascularization.

Reports generated by processing the fusion image data set can include, for example, automated suggestions of guidewire caliber based on the diameter of a recommended path for an interventional procedure; automated suggestion of wire stiffness based on the identification of a completely occlusive hard lesion component that would require specialized stiff wires; automated identification of the eccentricity of hard lesion components; centerline measurements and vessel diameter measurements for appropriate sizing of balloons and stents and both proximal and distal ends; and automated device selection suggestions based on mechanical properties and morphology of the detected lesions.

In some implementations of the methods described in the present disclosure, spatial resolution is significantly reduced as compared to high resolution imaging that is capable in ex vivo samples (e.g., 1×1×1 mm³ versus 0.75×0.75×0.75 μm). Particularly, although higher resolutions are possible using clinical MRI scanners, it may not be practically feasible to further increase the spatial resolution because of the necessary trade-off of requiring longer scan times. However, there is an advantage to using clinical scanners with coarser spatial resolution because a coarser spatial resolution significantly improves signal-to-noise ratio (“SNR”). SNR is proportional to voxel volume and the square root of acquisition time. A coarser spatial resolution can therefore increase voxel volume significantly, so that even with the reduction in scan time the overall SNR improves by a significant factor. This increase in SNR can be exploited, as described above, by performing image subtraction, which relies on adequate SNR. Combination of first and second images as described above provided sufficient contrast and retained sufficient image quality to accurately analyze hard lesion components.

The methods described in the present disclosure can be used to distinguish hard and peripheral artery disease lesions (e.g., densely calcified or collagenous lesions) from soft lesions. Hard lesions would be at high risk of PVI failure, whereas soft lesions would be amenable to PVI. These methods benefit the planning of interventional procedures, such as by reducing PVI failure rates, reducing time to definitive revascularization, and reducing costs for additional procedures and investigations.

The methods described in the present disclosure are described with respect to planning peripheral endovascular procedures. Personas having ordinary skill in the art will appreciated, however, that the methods described in the present disclosure can also be used can inform planning other interventional procedures, including endovascular aneurysm repair, percutaneous coronary interventions, carotid stenting, organ biopsies (e.g., kidney, liver, thyroid), percutaneous drainage of cystic versus solid lesions, and so on. The methods described in the present disclosure also facilitate the assessment of angiosome perfusion, which can be useful for the surgical planning and follow-up assessment of microvascular reconstructions with tissue flaps.

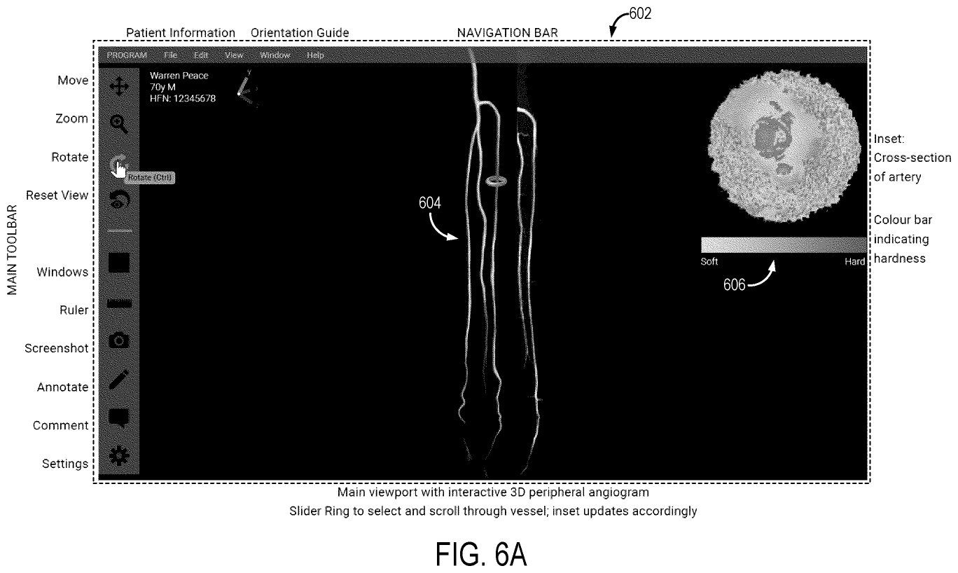

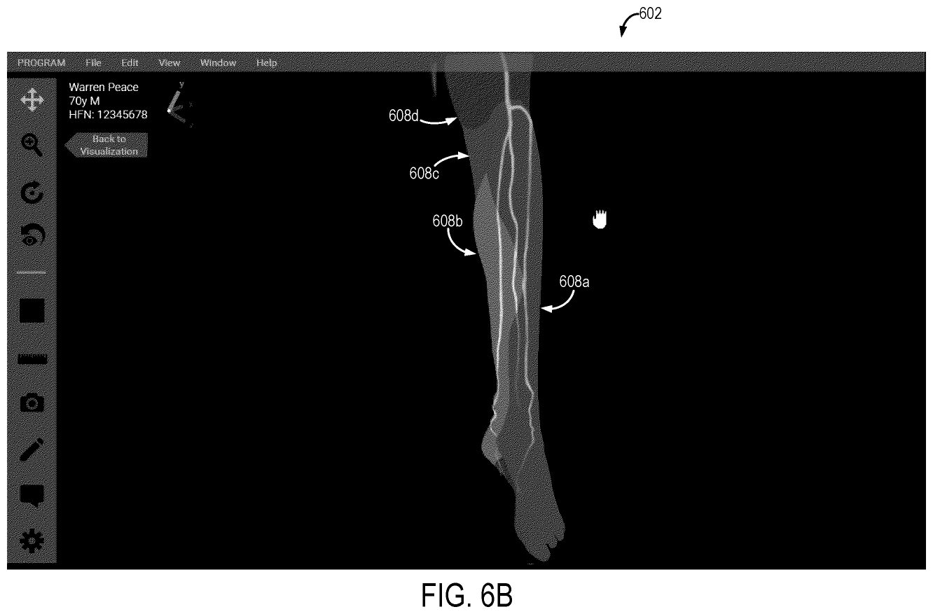

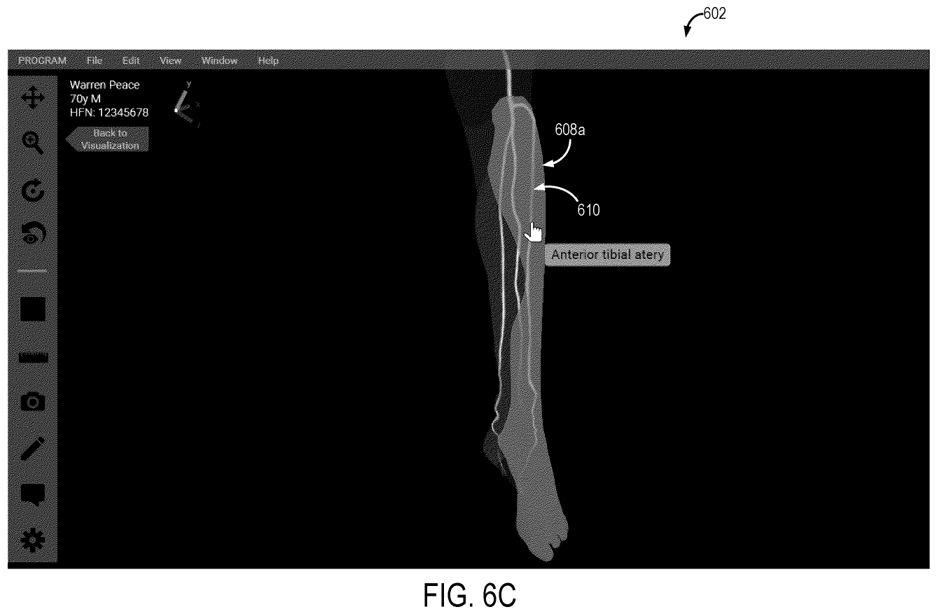

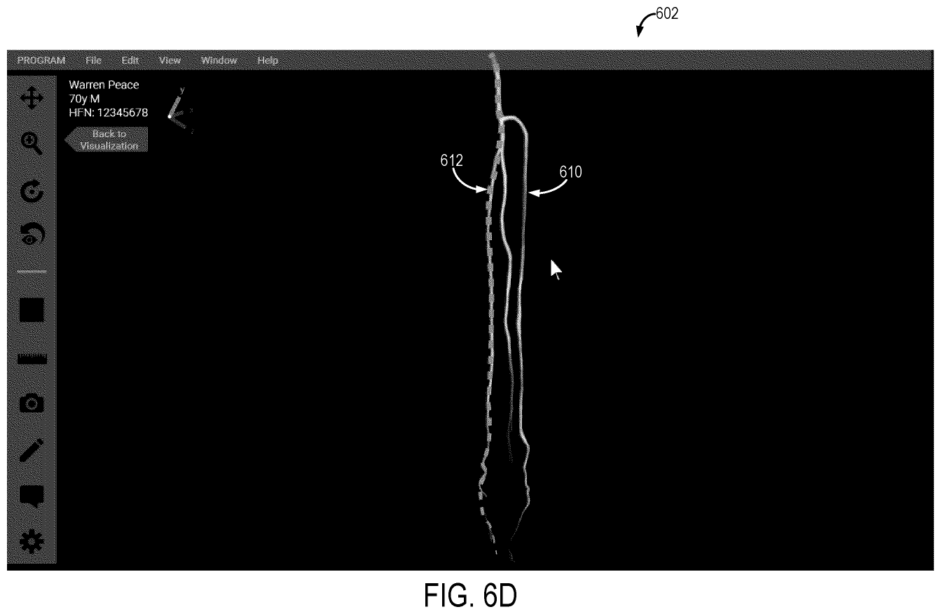

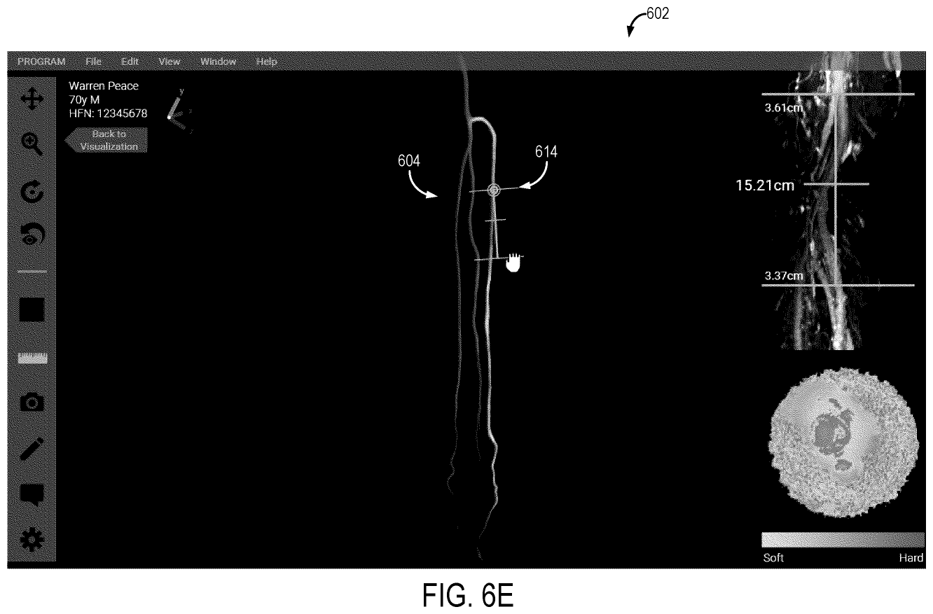

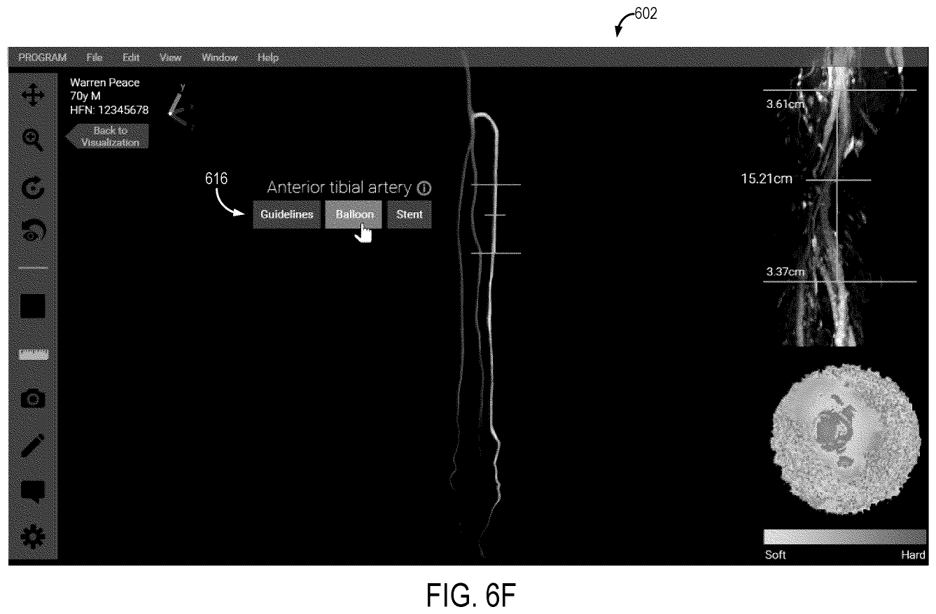

In some aspects of the present disclosure, a treatment planning application implemented with a hardware processor and memory is provided. The treatment planning application can include a user interface 602, as indicated in FIG. 6A, which in response to control input from a user can implement the methods described in the present disclosure. The user interface 602 can include a display of images, such as angiography images, UTE images, fused image data, angiograms, or other images. In the example of FIG. 6A, a three-dimensional angiogram 604 is shown as displayed. The user interface 602 also displays cross-sectional images through a displayed angiogram, as shown in FIG. 6A, and can display an indicator 606 that indicates the hardness of a lesion. The indicator 606 may be, for instance, a color scale.

As shown in FIG. 6B, the user interface 602 can also display angiosomes 608a-608d in a planning mode. The angiosomes can be panned, zoomed, and rotated along with the displayed angiogram. In response to user input, one or more of the displayed angiosomes can be selected. As shown in FIG. 6C, when an angiosome 608a is selected, the associated feeder artery 610 can also be highlighted or otherwise labeled. As shown in FIG. 6D, selecting an angiosome 608a can also provide a display of an alternative revascularization path 612 using the methods described in the present disclosure.

Based on a selection of the affected artery displayed in the user interface 602, a display inset can be generated and provided to the user interface to show the longitudinal section of the selected artery, as shown in FIG. 6E. In addition, a 614 tool can be provided to the user interface whereby the user can interact with the tool 614 to measure distances and diameters in the angiogram 604. As shown in FIG. 6F, the user can also open an intervention menu 616 that can implement the methods described in the present disclosure to calculate the most effective treatment method given the severity of the disease.

For instance, by analyzing the fusion image data set, a lesion and the surrounding vasculature can be characterized. If the lesion is soft and likely crossable, then the user interface 602 can generate and display an indication that the patient can be referred for PVI. If the lesion is hard and there is a suitable conduit and outflow vessel to bypass, then the user interface 602 can generate and display an indication that the patient can be referred to bypass surgery. If the patient is at high likelihood of endovascular failure and has no outflow vessels for bypass, then the user interface 602 can generate and display an indication that the patient can be referred for amputation of the affected limb.

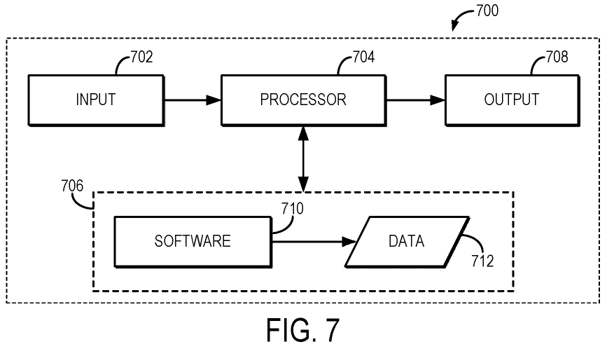

Referring now to FIG. 7, a block diagram of an example of a computer system 700 that can perform the methods described in the present disclosure is shown. The computer system 700 includes an input 702, at least one processor 704, a memory 706, and an output 708. The computer system 700 can also include any suitable device for reading computer-readable storage media. The computer system 700 may be implemented, in some examples, a workstation, a notebook computer, a tablet device, a mobile device, a multimedia device, a network server, a mainframe, or any other general-purpose or application-specific computing device.

The computer system 700 may operate autonomously or semi-autonomously, or may read executable software instructions from the memory 706 or a computer-readable medium (e.g., a hard drive, a CD-ROM, flash memory), or may receive instructions via the input 702 from a user, or any another source logically connected to a computer or device, such as another networked computer or server. In general, the computer system 700 is programmed or otherwise configured to implement the methods and algorithms described above.

The input 702 may take any suitable shape or form, as desired, for operation of the computer system 700, including the ability for selecting, entering, or otherwise specifying parameters consistent with performing tasks, processing data, or operating the computer system 700. In some aspects, the input 702 may be configured to receive data, such as magnetic resonance images, patient health data, and so on. Such data may be processed as described above to characterize lesion hardness, assess vessel occlusion or patency, generate a treatment plan for an interventional procedure, and so on. In addition, the input 702 may also be configured to receive any other data or information considered useful for characterizing lesion hardness, assessing vessel occlusion or patency, generating a treatment plan for an interventional procedure, and so on using the methods described above.

Among the processing tasks for operating the signal reconstruction unit 700, the at least one processor 704 may also be configured to carry out any number of post-processing steps on data received by way of the input 702.

The memory 706 may contain software 710 and data 712, such as magnetic resonance images, patient health data, and so on, and may be configured for storage and retrieval of processed information, instructions, and data to be processed by the at least one processor 704. In some aspects, the software 710 may contain instructions directed to implementing the methods described in the present disclosure.

In addition, the output 708 may take any shape or form, as desired, and may be configured for displaying, in addition to other desired information, reconstructed signals or images.

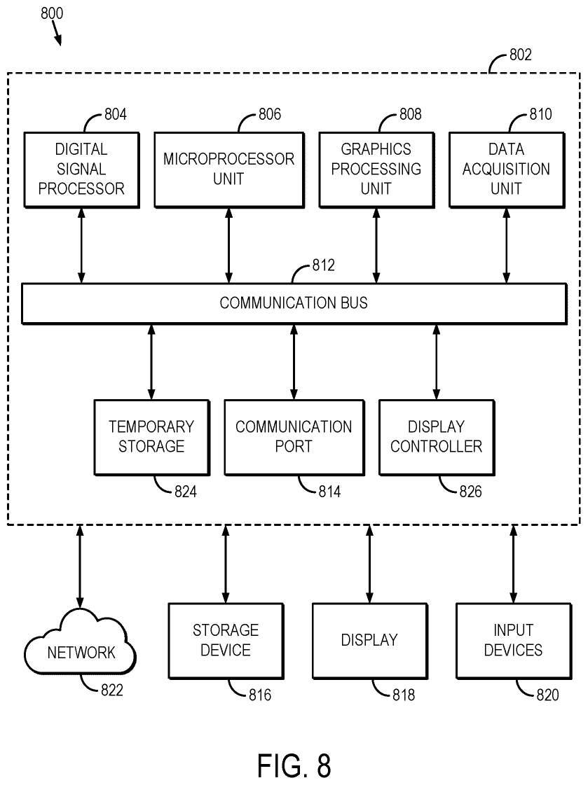

Referring now to FIG. 8, a block diagram of an example of another computer system 800 that can be configured to implement the methods described in the present disclosure is illustrated. Data, such as magnetic resonance images, can be provided to the computer system 800 from a data storage device, and these data are received in a processing unit 802.

In some embodiments, the processing unit 802 can include one or more processors. As an example, the processing unit 802 may include one or more of a digital signal processor (“DSP”) 804, a microprocessor unit (“MPU”) 806, and a graphics processing unit (“GPU”) 808. The processing unit 802 can also include a data acquisition unit 810 that is configured to electronically receive data to be processed. The DSP 804, MPU 806, GPU 808, and data acquisition unit 810 are all coupled to a communication bus 812. As an example, the communication bus 812 can be a group of wires, or a hardwire used for switching data between the peripherals or between any component in the processing unit 802.

The DSP 804 can be configured to implement the methods described here. The MPU 806 and GPU 808 can also be configured to implement the methods described here in conjunction with the DSP 804. As an example, the MPU 806 can be configured to control the operation of components in the processing unit 802 and can include instructions to implement the methods for characterizing lesion hardness, assessing vessel occlusion or patency, generating a treatment plan for an interventional procedure, and so on, on the DSP 804. Also as an example, the GPU 808 can process image graphics, such as displaying magnetic resonance images, fusion image data, reports generated based on such images or data, a user interface, and so on.

The processing unit 802 preferably includes a communication port 814 in electronic communication with other devices, which may include a storage device 816, a display 818, and one or more input devices 820. Examples of an input device 820 include, but are not limited to, a keyboard, a mouse, and a touch screen through which a user can provide an input.

The storage device 816 is configured to store data, which may include magnetic resonance images, whether these data are provided to or processed by the processing unit 802. The display 818 is used to display images and other information, such as magnetic resonance images, patient health data, and so on.

The processing unit 802 can also be in electronic communication with a network 822 to transmit and receive data and other information. The communication port 814 can also be coupled to the processing unit 802 through a switched central resource, for example the communication bus 812.

The processing unit 802 can also include a temporary storage 824 and a display controller 826. As an example, the temporary storage 824 can store temporary information. For instance, the temporary storage 824 can be a random access memory.

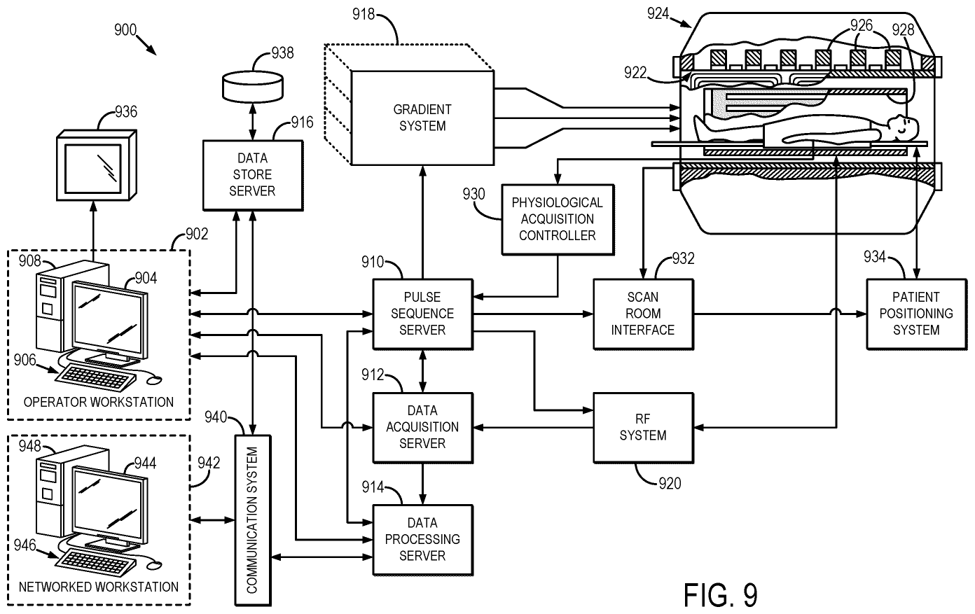

Referring particularly now to FIG. 9, an example of an MRI system 900 that can implement the methods described here is illustrated. The MRI system 900 includes an operator workstation 902 that may include a display 904, one or more input devices 906 (e.g., a keyboard, a mouse), and a processor 908. The processor 908 may include a commercially available programmable machine running a commercially available operating system. The operator workstation 902 provides an operator interface that facilitates entering scan parameters into the MRI system 900. The operator workstation 902 may be coupled to different servers, including, for example, a pulse sequence server 910, a data acquisition server 912, a data processing server 914, and a data store server 916. The operator workstation 902 and the servers 910, 912, 914, and 916 may be connected via a communication system 940, which may include wired or wireless network connections.

The pulse sequence server 910 functions in response to instructions provided by the operator workstation 902 to operate a gradient system 918 and a radiofrequency (“RF”) system 920. Gradient waveforms for performing a prescribed scan are produced and applied to the gradient system 918, which then excites gradient coils in an assembly 922 to produce the magnetic field gradients Gₓ, G_{y}, and G_{z} that are used for spatially encoding magnetic resonance signals. The gradient coil assembly 922 forms part of a magnet assembly 924 that includes a polarizing magnet 926 and a whole-body RF coil 928.

RF waveforms are applied by the RF system 920 to the RF coil 928, or a separate local coil to perform the prescribed magnetic resonance pulse sequence. Responsive magnetic resonance signals detected by the RF coil 928, or a separate local coil, are received by the RF system 920. The responsive magnetic resonance signals may be amplified, demodulated, filtered, and digitized under direction of commands produced by the pulse sequence server 910. The RF system 920 includes an RF transmitter for producing a wide variety of RF pulses used in MRI pulse sequences. The RF transmitter is responsive to the prescribed scan and direction from the pulse sequence server 910 to produce RF pulses of the desired frequency, phase, and pulse amplitude waveform. The generated RF pulses may be applied to the whole-body RF coil 928 or to one or more local coils or coil arrays.

The RF system 920 also includes one or more RF receiver channels. An RF receiver channel includes an RF preamplifier that amplifies the magnetic resonance signal received by the coil 928 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M≤\sqrt{I²+Q²} (1);

and the phase of the received magnetic resonance signal may also be determined according to the following relationship:

$$ϕ = {tan}^{- 1} ( \frac{Q}{I} ) . \qquad ( 2 )$$

The pulse sequence server 910 may receive patient data from a physiological acquisition controller 930. By way of example, the physiological acquisition controller 930 may receive signals from a number of different sensors connected to the patient, including electrocardiograph (“ECG”) signals from electrodes, or respiratory signals from a respiratory bellows or other respiratory monitoring devices. These signals may be used by the pulse sequence server 910 to synchronize, or “gate,” the performance of the scan with the subject's heart beat or respiration.

The pulse sequence server 910 may also connect to a scan room interface circuit 932 that receives signals from various sensors associated with the condition of the patient and the magnet system. Through the scan room interface circuit 932, a patient positioning system 934 can receive commands to move the patient to desired positions during the scan.

The digitized magnetic resonance signal samples produced by the RF system 920 are received by the data acquisition server 912. The data acquisition server 912 operates in response to instructions downloaded from the operator workstation 902 to receive the real-time magnetic resonance data and provide buffer storage, so that data is not lost by data overrun. In some scans, the data acquisition server 912 passes the acquired magnetic resonance data to the data processor server 914. In scans that require information derived from acquired magnetic resonance data to control the further performance of the scan, the data acquisition server 912 may be programmed to produce such information and convey it to the pulse sequence server 910. For example, during pre-scans, magnetic resonance data may be acquired and used to calibrate the pulse sequence performed by the pulse sequence server 910. As another example, navigator signals may be acquired and used to adjust the operating parameters of the RF system 920 or the gradient system 918, or to control the view order in which k-space is sampled. In still another example, the data acquisition server 912 may also process magnetic resonance signals used to detect the arrival of a contrast agent in a magnetic resonance angiography (“MRA”) scan. For example, the data acquisition server 912 may acquire magnetic resonance data and processes it in real-time to produce information that is used to control the scan.

The data processing server 914 receives magnetic resonance data from the data acquisition server 912 and processes the magnetic resonance data in accordance with instructions provided by the operator workstation 902. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 914 are conveyed back to the operator workstation 902 for storage. Real-time images may be stored in a data base memory cache, from which they may be output to operator display 902 or a display 936. Batch mode images or selected real time images may be stored in a host database on disc storage 938. When such images have been reconstructed and transferred to storage, the data processing server 914 may notify the data store server 916 on the operator workstation 902. The operator workstation 902 may be used by an operator to archive the images, produce films, or send the images via a network to other facilities.

The MRI system 900 may also include one or more networked workstations 942. For example, a networked workstation 942 may include a display 944, one or more input devices 946 (e.g., a keyboard, a mouse), and a processor 948. The networked workstation 942 may be located within the same facility as the operator workstation 902, or in a different facility, such as a different healthcare institution or clinic.

The networked workstation 942 may gain remote access to the data processing server 914 or data store server 916 via the communication system 940. Accordingly, multiple networked workstations 942 may have access to the data processing server 914 and the data store server 916. In this manner, magnetic resonance data, reconstructed images, or other data may be exchanged between the data processing server 914 or the data store server 916 and the networked workstations 942, such that the data or images may be remotely processed by a networked workstation 942.

The present disclosure has described one or more preferred embodiments, and it should be appreciated that many equivalents, alternatives, variations, and modifications, aside from those expressly stated, are possible and within the scope of the invention.

1. A method for characterizing a lesion in a subject using magnetic resonance imaging (MRI), the steps of the method comprising:
(a) providing to a computer system:
magnetic resonance images acquired from a volume-of-interest in a subject;
first images acquired from the volume-of-interest in the subject using a first echo time that is in a range of ultrashort echo times;
second images acquired from the volume-of-interest in the subject using a second echo time that is longer than an ultrashort echo time;
(b) producing combined images by computing a mathematical combination of the first images and the second images;
(c) identifying a lesion in the magnetic resonance images; and
(d) characterizing mechanical properties of the identified lesion based at least in part on a comparison of magnetic resonance signal behaviors between the magnetic resonance images and the combined images.

2. The method as recited in claim 1, wherein the magnetic resonance images are acquired using a flow-independent angiography pulse sequence.

3. The method as recited in claim 2, wherein the non-contrast-enhanced angiography pulse sequence is a binomial pulse steady-state free precession (BP-SSFP) pulse sequence.

4. The method as recited in claim 1, wherein the first echo time is less than 1 millisecond.

5. The method as recited in claim 1, wherein identifying the lesion includes detecting regions of signal drop-out in the magnetic resonance images.

6. The method as recited in claim 1, wherein characterizing the identified lesion includes characterizing soft lesion components based on the magnetic resonance images and characterizing hard lesion components based on the combined images.

7. The method as recited in claim 6, further comprising generating fusion image data based on the magnetic resonance images and the combined images, wherein the fusion image data provides a visual depiction of the soft lesion components and the hard lesion components.

8. The method as recited in claim 1, wherein characterizing the identified lesion includes plotting signal intensities in the magnetic resonance images and the combined images against each other and inputting the plotted signal intensities to a classifier.

9. The method as recited in claim 1, wherein the computing the mathematical combination of the first images and the second images comprising computing a linear combination of the first images and the second images.

10. The method as recited in claim 9, wherein the linear combination is one of a difference or a weighted difference.

11. A method for generating an endovascular procedure plan using magnetic resonance imaging (MRI), the steps of the method comprising:
(a) providing to a computer system:
magnetic resonance angiography images acquired from a volume-of-interest in a subject;
first images acquired from the volume-of-interest in the subject using a first echo time that is in a range of ultrashort echo times;
second images acquired from the volume-of-interest in the subject using a second echo time that is longer than an ultrashort echo time;
(b) generating a three-dimensional angiogram from the magnetic resonance angiography images, the three-dimensional angiogram depicting a vasculature of the subject;
(c) producing combined images by computing a mathematical combination of the first images and the second images;
(d) identifying a lesion in the magnetic resonance angiography images;
(e) generating fusion image data based on a combination of the magnetic resonance angiography images and the combined images, wherein the fusion image data provides a characterization of the identified lesion; and
(f) processing the fusion image data and the three-dimensional angiogram to generate a report that indicates an endovascular procedure plan for the subject.

12. The method as recited in claim 11, wherein processing the fusion image data and the three-dimensional angiogram includes characterizing soft lesion components in the identified lesion based on the magnetic resonance angiography images and characterizing hard lesion components in the identified lesion based on the combined images.

13. The method as recited in claim 12, wherein the generated report indicates an eccentricity of the hard lesion components.

14. The method as recited in claim 12, wherein the generated report indicates a tool selection for treating the identified lesion based on the characterized soft lesion components and hard lesion components of the identified lesion.

15. The method as recited in claim 14, wherein the tool selection includes a stiffness of a wire.

16. The method as recited in claim 11, wherein processing the fusion image data and the three-dimensional angiogram includes analyzing the three-dimensional angiogram to determine at least one of centerline measurements and vessel diameter measurements for vessels in the vasculature of the subject.

17. The method as recited in claim 16, wherein the generated report indicates at least one of occlusion or patency based at least in part on the determined at least one of centerline measurements and vessel diameter measurements for vessels in the vasculature of the subject.

18. The method as recited in claim 16, wherein the generated report indicates at least one pathway through the vasculature of the subject for treating the identified lesion.

19. The method as recited in claim 18, wherein the generated report indicates a tool selection for treating the identified lesion based on the at least one pathway through the vasculature of the subject.

20. The method as recited in claim 19, wherein the tool selection includes at least one of a guide wire caliber, a balloon size, or a stent size.

21. The method as recited in claim 11, wherein processing the fusion image data and the three-dimensional angiogram includes identifying at least one angiosome in the subject based at least in part on the fusion image data and the three-dimensional angiogram.

22. The method as recited in claim 21, wherein the generated report includes a visual depiction of the at least one angiosome including an indication of a feeding artery for the at least one angiosome.

23. The method as recited in claim 11, wherein the computing the mathematical combination of the first images and the second images comprising computing a linear combination of the first images and the second images.

24. The method as recited in claim 23, wherein the linear combination is one of a difference or a weighted difference.
